(19) United States
(12) Patent Application Publication
(10) Pub. No.: US 2009/0326635 A1
(43) Pub. Date: Dec. 31, 2009

(54) Arterial Endoprosthesis

(75) Inventor: Nicolas Durrleman, Paris (FR)

Correspondence Address: MATTHEW R. JENKINS, ESQ., 2310 FAR HILLS BUILDING, DAYTON, OH, 45419, US

(73) Assignee: ASSOCIATION RENE LERICHE, PARIS (FR)

(21) Appl. No.: 12/087,490

(22) PCT Filed: Jan. 11, 2007
(86) PCT No.: PCT/FR2007/050639

§ 371 Date: Nov. 10, 2008

(30) Foreign Application Priority Data
Jan. 11, 2006 (FR) 0600235

(51) Int. Cl. A61F 2/06

(52) U.S. Cl. 623/1.15

(57) ABSTRACT

An arterial endoprosthesis, in particular for the aorta, having an outer face designed to press against the inner wall of an artery and including, on its outer face, projections distributed in such a manner as to reduce the speed of a flow of blood passing between the artery and the prosthesis.

### CROSS-REFERENCE TO RELATED APPLICATIONS

This application is the U.S. National Phase application of PCT/FR2007/050639 filed Jan. 11, 2007, which claims priority to French Application No. 0600235 filed Jan. 11, 2006, which applications are incorporated herein by reference and made a part hereof.

### BACKGROUND OF THE INVENTION

I. Field of the Invention

The present invention relates to an arterial endoprosthesis, in particular for the aorta.

The aorta is an artery of the human body going from the left ventricle of the heart, down along the spinal column, and from which numerous blood vessels originate that irrigate the entire body.

Prostheses for the aorta are commonly used in the context of surgery. They are capable of reducing the risk of an aneurysm rupturing, where an aneurysm is a non-natural and localized expansion of the aorta, which can often be the cause of sudden death.

II. Description of the Related Art

In the prior art, there is known in particular a tubular endoprosthesis for the aorta that comprises a leakproof duct situated inside the aorta. This is shown in FIG. 1.

The leakproof duct 12 matches the diameter of the aorta 4 with the help of a spring 14 placed inside the duct so as to provide a path for blood flow in register with the aneurysm 6, this path having the usual diameter for the aorta.

The object of that type of endoprosthesis 2 is to prevent blood from flowing in the expanded portion of the aneurysm 6. Nevertheless, in spite of the pressure exerted by the spring 4 on the duct 12 so that it adheres to the wall 16 and makes a leaktight connection 17 between the wall and the prosthesis, a flow of blood 18 can pass outside the duct, because of the pressure exerted by the blood against the walls 16.

This flow of blood 18 passing through the expanded portion of the aneurysm as a result of incomplete contact between the prosthesis 2 and the wall 16 is a phenomenon referred to as “type 1 endoleak”.

These leaks create pressure against the wall 16 of the aneurysm 6, meaning that it is not possible to completely eliminate any risk of additional expansion, and thus of rupture.

### SUMMARY OF THE INVENTION

A particular object of the invention is to remedy those drawbacks by providing an aortic endoprosthesis capable of eliminating any risk of type 1 endoleaks, and thus of reducing the failure rate of surgery for putting a prosthesis into place.

To this end, the invention provides an arterial endoprosthesis having an outer face for pressing against the inside wall of an artery, the endoprosthesis being characterized in that it includes, on its outer face, projections that are distributed in such a manner as to reduce the speed of a flow of blood passing between the artery and the endoprosthesis.

In their vicinity, the projections create turbulence that disturbs the flow and slows it down. This encourages thrombosis or blood coagulation. The blood coagulated in this way can then contribute to plugging the orifice through which the blood has passed outside the duct, thus preventing endoleaks.

Furthermore, because of the presence the projections, the roughness of the surface is considerable, thereby providing better adhesion for the prosthesis with the wall of the artery than is provided with existing models, thus avoiding possible migration of the prosthesis.

In addition, the endoprosthesis of the invention makes it possible to avoid type 3 endoleaks that occur in the event of a microtear in the duct of the prosthesis. The coagulated blood then prevents fluid blood that is flowing inside the duct from escaping through the microleak.

In a particular embodiment, the projections are hemispherical in shape.

In a particular embodiment, the projections are disposed in a staggered configuration along the longitudinal direction of the endoprosthesis. This optimizes the slowing down of the flow.

Advantageously, the projections are made of microporous material. Such projections absorb a fraction of the blood flowing in the expanded portion of the artery. The blood retained in the projections and thus prevented from moving coagulates quickly. Blood coagulation takes place progressively, so the blood that has coagulated in the projections initiates coagulation of the blood that has been slowed down and that is flowing between them.

Coagulation thus takes place more quickly since it is initiated at a plurality of locations simultaneously.

Advantageously, the outer wall is covered in a film of leakproof material, which film dissolves slowly in contact with blood, thereby enabling the projections to retain a minimum volume during implantation.

The projections are then covered in a leakproof film, and during the first few hours that the prosthesis is present in the human body, the absorption function of the projections is not activated.

Given that the prosthesis is generally inserted in the human body via the femoral artery and then slid up the artery to the aneurysm, it is made much easier to put into place by the fact that the projections are in compact form and of minimum volume. Because of the leakproof film, blood does not penetrate immediately into the projections, so they conserve a compact minimum volume. After the endoprosthesis has been in place for a few hours, the film dissolves away in contact with the blood, thereby enabling the microporous projections to act in the manner described above.

The film is optionally made of gelatin.

Other objects and advantages of the invention will be apparent from the following description, the accompanying drawings and the appended claims.

### BRIEF DESCRIPTION OF THE DRAWINGS

The invention can be better understood on reading the following description given purely by way of example and made with reference to the accompanying drawings, in which:

FIG. 1 is a view of a prior art arterial endoprosthesis, described above;

FIG. 2 is a side view of the endoprosthesis in a particular embodiment;

FIG. 3A is a longitudinal section of the endoprosthesis in the same embodiment, prior to being inserted in the human body; and

FIG. 3B is a longitudinal section of the endoprosthesis in the same embodiment, after it has been put into place at the location of the aneurysm.

### DETAILED DESCRIPTION OF THE INVENTION

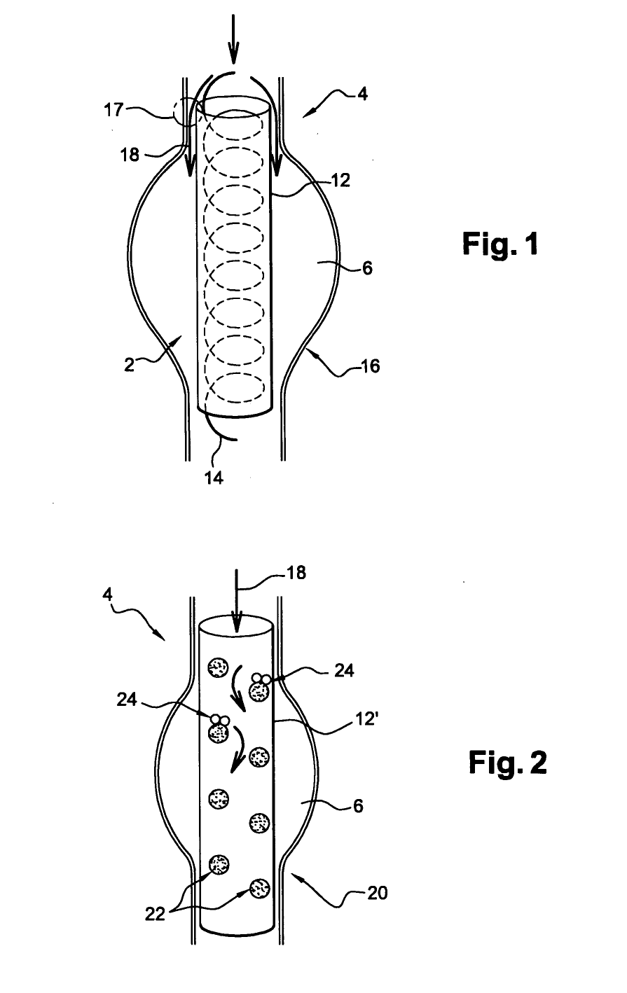

The figures show the endoprosthesis 20 of the invention. Like the endoprosthesis 2 known to the person skilled in the art and shown in FIG. 1, this endoprosthesis comprises a leakproof duct 12′ made of polyester or of polytetrafluoroethylene (PTFE) providing a blood flow path for blood at the location of the aneurysm 6, together with a spring 14′ that is generally made of a metal alloy that is not corrosive and that is not harmful to the human body, and that enables the duct 12′ to match the diameter of the aorta.

The arterial endoprosthesis 20 also includes, on its outer face, hemispherical projections 22 disposed in a staggered configuration in its long direction.

The projections 22 thus create a “labyrinth” effect: turbulence 24 occurs accidentally in a flow of blood 18 between the wall 16 of the aorta and the prosthesis 20 this turbulence 24 reduces the speed of the blood in the endoleak.

Blood is also absorbed by the projections 22, since they are made of a microporous material, e.g. of woven polyester yarn. The absorbed blood is prevented from moving, thereby encouraging it to coagulate and produce a thrombosis. The disturbed and slowed blood flowing in the vicinity of the projections 22 then coagulates easily in turn because of its slow speed and the presence of coagulated blood in the vicinity thereof, given that coagulation propagates by spreading gradually.

This coagulation of the blood thus serves to close off durably the orifice through which the flow of blood 18 becomes engaged accidentally into the expanded portion of the aneurysm 6.

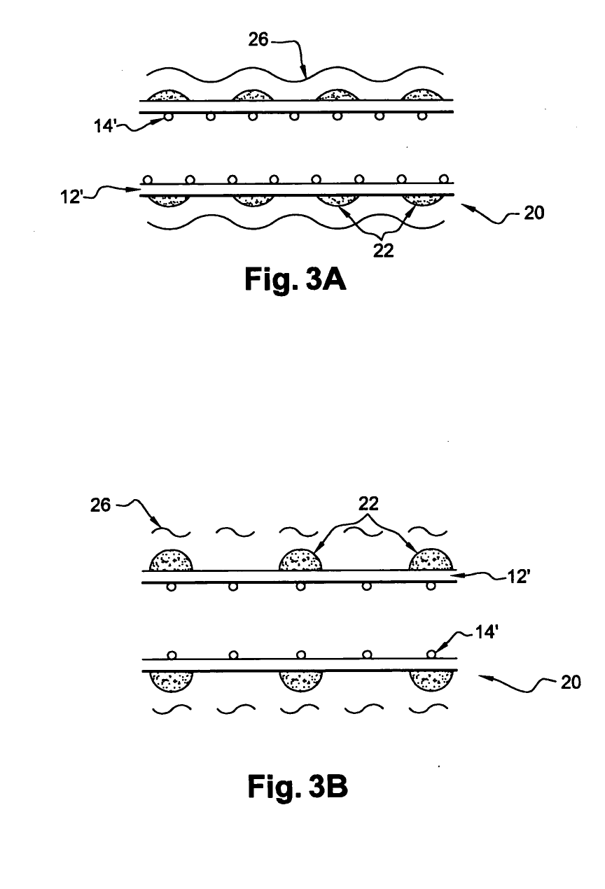

FIG. 3A shows the endoprosthesis of the invention before being inserted in the human body. The projections 22 are then covered in a leakproof film 26 of gelatin.

Thus, when the prosthesis is inserted into the human body via the femoral artery (leg artery), which is narrower than the aorta, the projections do not swell by absorbing blood: they are therefore compact and the volume of the prosthesis 20 is at a minimum. It is therefore easy to move the prosthesis up the femoral artery and the aorta 4 as far as the aneurysm 6.

Once the prosthesis is in place, the gelatin film 26, which is not harmful to the human body, dissolves into the blood, as shown in FIG. 3B, with this material dissolving slowly in contact with blood.

The projections 22 then come into contact with the flow of blood 18 and absorb the blood by the “sponge” effect. Consequently, they increase in volume by absorbing blood and they allow the blood to coagulate and obstruct the orifice, in the manner described above.

The projections 22 as swollen in this way after the endoprosthesis 20 has been put into place also serve to consolidate the positioning of the endoprosthesis by a “spike” effect. By increasing the roughness of the outer surface of the duct 12′, the swollen projections 22 provide better adhesion between the endoprosthesis 20 and the wall, and make any movement of the prosthesis difficult.

The endoprosthesis 20 as described above thus presents numerous advantages, enabling the risks of the aneurysm rupturing after surgery to be reduced, and more particularly eliminating certain types of endoleak.

Nevertheless, it will be understood that the invention is not limited to the above-described embodiments.

What is claimed is:

1. An arterial endoprosthesis having an outer face for pressing against the inside wall of an artery, said arterial endoprosthesis comprising, on its outer face, projections that are distributed in such a manner as to reduce the speed of a flow of blood passing between the artery and said arterial endoprosthesis.

2. The arterial endoprosthesis according to claim 1, in which said projections are hemispherical in shape.

3. The arterial endoprosthesis according to claim 1, in which said projections are disposed in a staggered configuration along a longitudinal direction of said arterial endoprosthesis.

4. The arterial endoprosthesis according to claim 1, in which said projections are made of microporous material.

5. The arterial endoprosthesis according to claim 1, in which the outer face is covered in a film of leakproof material, which film dissolves slowly in contact with blood, thereby enabling said projections to retain a minimum volume during implantation.

6. The arterial endoprosthesis according to claim 5, in which the film is made of gelatin.

7. An aortic endoprosthesis constituted by an arterial endoprosthesis according to claim 1.
